(19) United States
(12) Patent Application Publication
(10) Pub. No.: US 2026/0097091 A1
(43) Pub. Date: Apr. 9, 2026

(54) ADMINISTRATION OF GLUTATHIONE TRISULFIDE TO AMELIORATE PERIPHERAL NEUROPATHY

(71) Applicants: The General Hospital Corporation, Boston, MA (US); Kyowa Hakko Bio Co., Ltd., Tokyo (JP)

(72) Inventors: Fumito Ichinose, III, Chestnut Hill, MA (US); Mariko Ezaka, Tokyo (JP); Etsuo Ohshima, Nagareyama, Chiba (JP)

(21) Appl. No.: 19/113,849

(22) PCT Filed: Sep. 26, 2023
(86) PCT No.: PCT/US2023/075131

§ 371 Date: Mar. 20, 2025

Related U.S. Application Data
(60) Provisional application No. 63/377,379, filed on Sep. 28, 2022.
(60) Provisional application No. 63/379,298, filed on Oct. 13, 2022.

(51) Int. Cl. A61K 38/06; A61K 31/16; A61K 31/385; A61P 25/02

(52) U.S. Cl. CPC A61K 38/063; A61K 31/16; A61K 31/385; A61P 25/02

(57) ABSTRACT

Methods and devices for the administration of compositions comprising glutathione trisulfide (GSSSG), pantethine trisulfide (PTN-SSS), or lipoic acid trisulfide (LA-SSS) to treat peripheral neuropathy, e.g., chemotherapy-induced peripheral neuropathy (CIPN), e.g., by oral or nasal administration. The methods can be used, e.g., to reduce pain associated with CIPN, or reduce the risk of development of CIPN.

### CLAIM OF PRIORITY

This application claims the benefit of U.S. Provisional Application Ser. No. 63/377,379, filed on Sep. 28, 2022; and 63/379,298, filed on Oct. 13, 2022. The entire contents of the foregoing are incorporated herein by reference.

### SEQUENCE LISTING

This application contains a Sequence Listing that has been submitted electronically as an XML file named “29539-0707WO1_SL_ST26.XML.” The XML file, created on Sep. 25, 2023, is 9,860 bytes in size. The material in the XML file is hereby incorporated by reference in its entirety.

### TECHNICAL FIELD

Methods and devices for the administration of compositions comprising glutathione trisulfide (GSSSG), pantethine trisulfide (PTN-SSS), or lipoic acid trisulfide (LA-SSS) to treat peripheral neuropathy, e.g., chemotherapy-induced peripheral neuropathy (CIPN), e.g., by oral or nasal administration. The methods can be used, e.g., to reduce pain associated with CIPN, or reduce the risk of development of CIPN.

### BACKGROUND

As of 2018, 9.8 million cancer patients were treated with chemotherapy, and the number of patients requiring cancer chemotherapy is expected to reach 15 million in 2040 [1]. Paclitaxel (PTX) is one of the most common chemotherapy drugs used to treat breast, ovarian, and prostate cancer. Paclitaxel-induced peripheral neuropathy (PIPN) is a dose-limiting side effect of paclitaxel, affecting 30% to 70% of patients treated with paclitaxel [2].

PIPN manifests as allodynia, hyperalgesia and spontaneous pain, predominantly involving feet and hands [48]; PIPN generally develops during chemotherapy and often persists after cessation of paclitaxel [3]. PIPN manifests as mechanical allodynia and hyperalgesia predominantly involving feet and hands during chemotherapy and often persists even after cessation of chemotherapy [3]. PIPN worsens the quality of life of cancer survivors and can be severe enough to lead to discontinuation of chemotherapy. Unfortunately, other chemotherapy drugs including cisplatin and vincristine can also cause peripheral neuropathy of similar clinical symptoms and shared pathogenetic mechanisms; inclusively termed as chemotherapy-induced peripheral neuropathy (CIPN) [4]. Because of the growing number of cancer survivors worldwide [1], the medico-economical impact of CIPN has increased significantly. Currently, there are very limited treatment options available for CIPN, including PIPN.

### SUMMARY

Provided herein are methods for the treatment, or reduction of risk, of a disorder associated with peripheral neuropathy in a subject, the method comprising administering, e.g., orally or nasally administering, a therapeutically or prophylactically effective amount of a composition comprising glutathione trisulfide (GSSSG), pantethine trisulfide (PTN-SSS), or lipoic acid trisulfide (LA-SSS) to a subject in need thereof. Also provided herein are compositions for administration, e.g., oral or nasal administration, comprising Glutathione Trisulfide (GSSSG), pantethine trisulfide (PTN-SSS), or lipoic acid trisulfide (LA-SSS) for use in the treatment, or reduction of risk, of a disorder associated with peripheral neuropathy in a subject, the method comprising administering, e.g., orally or nasally administering, a therapeutically or prophylactically effective amount of to a subject in need thereof, optionally wherein the composition is formulated for administration orally or nasally.

In some embodiments, the composition comprising GSSSG is prepared by dissolving a crystalline form of GSSSG in buffered saline at pH 3-6.

In some embodiments, the disorder is chemotherapy induced peripheral neuropathy (CIPN). In some embodiments, the CIPN is peripheral neuropathy associated with administration of another platinum-containing chemotherapy, e.g., paclitaxel, cisplatin, vincristine, carboplatin, oxaliplatin, or nedaplatin, e.g., paclitaxel-induced peripheral neuropathy (PIPN). In some embodiments, the method comprises administering an effective amount of a composition comprising GSSSG, PTN-SSS, or LA-SSS within a few minutes to hours before or after administration of a dose of chemotherapy. In some embodiments, the method comprises administering an effective amount of a composition comprising GSSSG, PTN-SSS, or LA-SSS every day for the first one or two weeks of a chemotherapy regimen.

In some embodiments, the disorder is diabetic neuropathy, Guillan-Barré syndrome neuropathy, chronic inflammatory demyelinating polyneuropathy, post herpetic neuralgia, or peripheral neuropathy caused by hepatitis B, hepatitis C, myeloma, lymphoma, or kidney disease.

Provided herein are methods of treating peripheral neuropathy in a patient comprising orally administering a therapeutically effective amount of a persulfide to the patient. In some embodiments, the peripheral neuropathy in the patient is a diabetic neuropathy, rheumatoid arthritis neuropathy, Guillan-Barre syndrome neuropathy, chronic inflammatory demyelinating polyneuropathy, or a neuropathy caused by Lyme disease, shingles (ie, post hepatic neuralgia), hepatitis B, hepatitis C, myeloma, lymphoma, or kidney disease. In some embodiments, the peripheral neuropathy in the patient is induced by chemotherapeutic drugs. In some embodiments, the drugs are paclitaxel, cisplatin or vincristine. In some embodiments, the persulfide is GSSSG. In some embodiments, the GSSSG is administered to the patient in a range of 25-100 mg per kg daily for 20-35 days. In some embodiments, the GSSSG is administered to the patient in an amount resulting in plasma level of GSSSG in the range of 40-80 pmol per ml for a period of 20-35 days.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-C. Chemical structures of polysulfides. (A). Chemical structures of GSSSG and GSH. Purified GSSSG is white, odorless solid powder at 20° C. Molecular weight is 644.7. One molecule of GSSSG consists of one molecule of sulfane sulfur (arrow) and two molecules of GSH. (B). Chemical structures of PTN-SSS and PTN. One molecule of PTN-SSS consists of one molecule of sulfane sulfur (arrow) and one molecule of PTN. (C.) Chemical structure of LA-SSS and LA. One molecule of LA-SSS consists of one molecule of sulfane sulfur and one molecule of LA. Abbreviations: GSH, glutathione; GSSSG, glutathione trisulfide; PTN, pantethine; PTN-SSS, pantethine trisulfide. LA-SSS. Lipoic acid trisulfide.

FIGS. 2A-B. GSSSG prevented mechanical allodynia evoked by paclitaxel-induced peripheral neuropathy. (A) Mechanical withdrawal threshold by the von Frey test for 4 weeks after paclitaxel treatment with or without co-treatment of GSSSG. 50 mg/kg of GSSSG prevented mechanical allodynia over the experimental period. (B) Relative change of response time compared to the baseline in hot plate test for 4 weeks. PTX induced thermal hyperalgesia. Data are analyzed by mixed effect model and adjusted by Bonferroni correction. Data are shown as the mean±SD, n=6 mice per group. PTX, paclitaxel.

FIGS. 3A-C. (A) Representative immunofluorescence images of intraepidermal nerve fibers (stained by PGP9.5, shown by yellow arrowheads) and basement membrane (stained by Collagen IV, white dashed lines) in hind paw at 1 week and 4 weeks after paclitaxel treatment. Quantification of unmyelinated fiber density calculated by dividing number of intraepidermal nerve fibers by basement membrane at 1 week (B) and at 4 weeks (C). Data were analyzed by one-way ANOVA with Dunnett's multiple comparisons test. Data are shown as the mean±SD, n=6 mice per group. PTX, paclitaxel; G, GSSSG.

FIGS. 4A-D. Neither PTX nor GSSSG changed number of myelinated axons. (A) Representative microscopic images of sciatic nerve at 4 weeks after PTX treatment stained with toluidine blue. (B) Quantification of total myelinated neurons axons in sciatic nerve. (C) Representative high magnification images of myelinated neurons axons to measure thickness of myelin. Ten neurons per image were randomly selected. The Calculation was achieved by GRatio software, an ImageJ plugin. (D) Quantification of G-ratios. Neither PTX nor GSSSG did not change thickness of myelin. Data were analyzed by one-way ANOVA with Dunnett's multiple comparisons test. Data are shown as the mean±SD, n=6 mice per group. PTX, paclitaxel; G, GSSSG.

FIGS. 5A-B. GSSSG prevented loss of unmyelinated axons in sciatic nerve. (A) Representative transmission electron microscopy images of sciatic nerve at 4 weeks. Number of unmyelinated axons (arrows) and myelinated axons (arrowheads) were counted. (B) Ratio of the number of unmyelinated axons of the total number of axons in sciatic nerve at 4 weeks. Data were analyzed by one-way ANOVA with Tukey's multiple comparisons test. Data are shown as the mean±SD, n=3 mice per group. PTX, paclitaxel. G, GSSSG.

FIGS. 6A-B. GSSSG prevented degeneration of mitochondria. (A) Representative transmission electron microscopy images of sciatic nerve at 4 weeks after paclitaxel treatment. Asterisks show Schwann cell, arrows indicate mitochondria and arrowheads indicate swollen mitochondria. (B) Quantification of mitochondria area in unmyelinated neuron. The median area of mitochondria in control, paclitaxel, and paclitaxel with GSSSG were 0.036 μm², 0.057 μm², and 0.041 μm², respectively. Red bars indicate median values of each group. Data were analyzed by Kruskal-Wallis test with Dunn's multiple comparisons test. Four high (×11000) magnification images from each mouse were examined. n=3 mice per group. PTX, paclitaxel; G, GSSSG.

FIGS. 7A-D. ³⁴S-labeled GSSSG and related polysulfides were detected in peripheral tissues 2 hours after oral administration of ³⁴S-labeled GSSSG. Quantification of GSSSG and reactive sulfur species by LC-MS/MS. (A) ³⁴S-labeled GSSSG concentration in 4 tissues that were obtained 2 hours after oral administration. Relative ratio of ³⁴S-labeled reactive sulfur species to endogenous reactive sulfur species (³²S-) were calculated. The ratios of ³⁴S-labeled to endogenous glutathione persulfide (GSSH) (B), cysteine persulfide (CysSSH) (C), cysteine trisulfide (CysSSSCys) (D) in lumber DRG, lumber spinal cord, brain, and liver were reported. Data are shown as the mean±SD, n=4 per group; DRG, dorsal root ganglion; LSC, lumber spinal cord.

FIGS. 8A-E. GSSSG prevented axon degeneration in primary DRG neurons. (A-B) Representative immunofluorescence images of primary DRG neurons stained by NF200. Neurons were cultured for 24 hours and incubated by paclitaxel with or without GSSSG (A) or PTNSSS (B) for one hour. (C) Sholl circles of cultured neurons from (A). The interval between Sholl circles is 10 μm. (D-E) Analysis of neuronal intersections with Sholl circles after treatment with paclitaxel with or without GSSSG (D) or PTNSSS (E) for one hour. Data were analyzed by repeated ANOVA with Dunnett's multiple comparisons test. P values are shown to indicate the comparison between paclitaxel and paclitaxel+GSSSG. Data are shown as the mean±SD, n=3 mice per group, 9-10 neurons per group. PTX, paclitaxel.

FIGS. 9A-C. GSSSG protected mitochondria in axon of primary DRG neurons. (A) Representative images of neural axons stained by NF200 with axonal mitochondria stained by MitoTracker. The number and length of mitochondria (yellow arrowheads) in axon was measured manually. (B) Ratio of the total mitochondria length to the total axonal length and (C) The number of MitoTracker puncta per 100 μm of axon. Data were analyzed by one-way ANOVA with Dunnett's multiple comparison. Data are shown as the mean±SD, n=3 mice per group, 8 images per group, and 450-790 mitochondria per group. PTX, paclitaxel; G, GSSSG.

FIG. 10. GSSSG treatment blocked increase of superoxide. Relative fluorescence change of Dihydroethidium (DHE) 30 minutes incubation of GSSSG and 60 minutes after paclitaxel exposure. Data were analyzed by one-way ANOVA with Dunnett's multiple comparisons test. Data are shown as the mean±SD, n=8 per group. PTX, paclitaxel; G, GSSSG.

FIGS. 11A-D. GSSSG upregulated antioxidant signaling in DRG. Relative gene expression of Nrf2 (A), HO1 (B), NQO1 (C), and GCLC (D) in DRG tissue at 2 hours after single 16 mg/kg paclitaxel with or without 50 mg/kg GSSSG treatment. Data were analyzed by one-way ANOVA with Dunnett's multiple comparisons test. Data are shown as the mean±SD, n=8 mice per group. PTX, paclitaxel; G, GSSSG; Nrf2, Nuclear factor-erythroid factor 2-related factor 2; HO1, Heme Oxygenase 1; NQO1, NAD (P) H Quinone Dehydrogenase 1; GCLC, Glutamate-Cysteine Ligase Catalytic Subunit.

FIGS. 12A-B. Co-administration of GSSSG did not inhibit anti-tumor effect of paclitaxel. (A) Cell viability of human breast cancer cell line, MDA-MB-231 measured by LDH assay at 24 hours after PTX with/without 10 μM of GSSSG. (B) Relative viable numbers of MDA-MB-231 cells 24 hours after 2 μM of PTX with/without 10 μM of GSSSG. Data were analyzed by two one-sided t-tests. The margin of equivalence was defined as 10% difference in cell count. Data are shown as the mean±SD, n=5 per group; LDH, lactate dehydrogenase, PTX, paclitaxel; G, GSSSG.

### DETAILED DESCRIPTION

While the precise mechanism of PIPN remains elusive, a pathological locus is the axon of peripheral sensory neurons [5-7], which explains why longer axons of sensory neurons in hands and feet are predominantly affected. Impairment of Aβ fibers leads to mechanical allodynia, while hyperalgesia arises from damaged Aδ and unmyelinated C fibers that transmit thermal and cold sensations, respectively [8]. Paclitaxel prevents cancer cell proliferation by stabilizing microtubule polymer that needs to be disassembled during mitosis. In addition to this anti-tumor effect, paclitaxel has been shown to cause mitochondrial dysfunction which manifests as mitochondrial swelling [9], decrease of mitochondrial membrane potential [10, 11], elevation of reactive oxygen spices (ROS) [12], and impaired oxidative phosphorylation in peripheral neurons [13]. These off-target effects of paclitaxel have been implicated in the pathogenesis of PIPN. Nonetheless, there are no therapeutic methods to protect mitochondria in axons of peripheral neurons.

Reactive sulfur species including persulfides and polysulfides contain reactive sulfur that oxidize or reduce other molecules. Especially, sulfane sulfur (S⁰) atom, one of reactive sulfur, has strong nucleophilicity that promotes persulfidation of protein thiols (cysteine residue). Thiol persulfidation competes with ROS-mediated thiol oxidation, thereby protecting proteins from irreversible oxidation. In fact, endogenous persulfides such as glutathione persulfide (GSSH) and cysteine persulfide (CysSSH) exert potent antioxidant effects, playing a key role in maintaining intracellular redox balance [14]. Nonetheless, whether systemic administration of polysulfides protects peripheral sensory neurons from injurious effects of paclitaxel by increasing local concentrations of reactive sulfur is unknown.

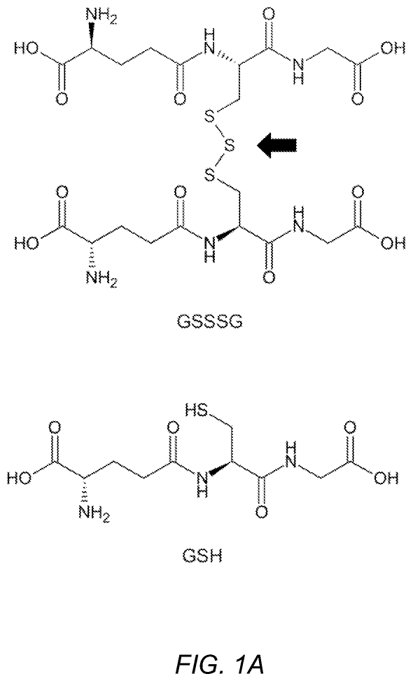

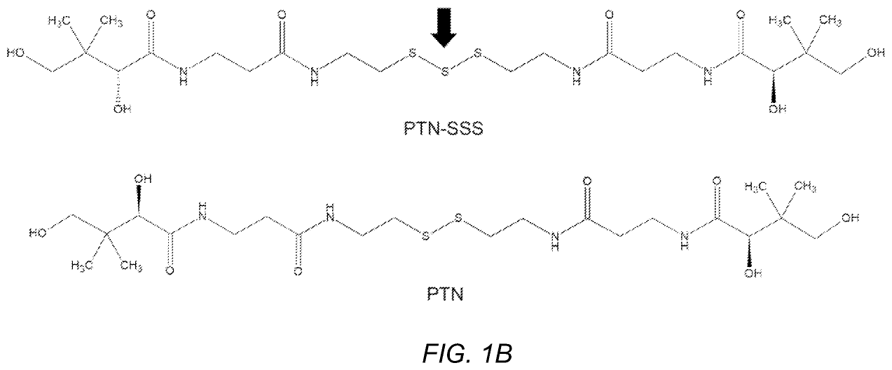

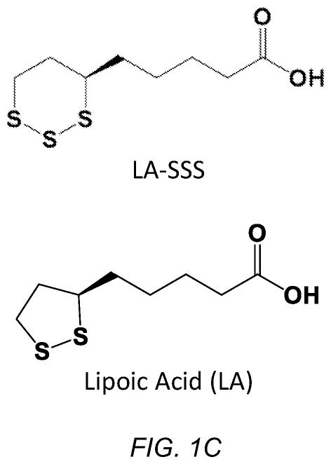

The present study was designed to address this knowledge gap by examining effects of systemically administered stable formulation of glutathione trisulfide (GSSSG), an endogenous polysulfide, in a mouse model of PIPN. GSSSG contains sulfane sulfur in the structure (FIG. 1A, arrow), and it is in an equilibrium between GSSH in the following equation [15].

$$GSSSG + GSH ↔ GSSH + GSSG \qquad ( Reaction 1 )$$

Without wishing to be bound by theory, it was hypothesized that systemic administration of GSSSG would increase local concentrations of reactive sulfur species in peripheral sensory nerves and ameliorate PIPN by protecting mitochondria in neural axons. As shown herein, GSSSG attenuates PIPN without reducing the anti-tumor effects of paclitaxel.

The current study revealed that polysulfides such as GSSSG have the potential to mitigate paclitaxel-induced peripheral neuropathy by protecting mitochondria in the axons of peripheral neurons. This conclusion is based on the following results: 1) daily oral administration of GSSSG attenuated mechanical allodynia in the hind paw of mice treated with paclitaxel; 2) orally-administered GSSSG was absorbed and increased reactive sulfur levels in lumbar DRG and spinal cord (both regions which contain the primary sensory neurons that innervate the hind paw); 3) GSSSG inhibited axonal degeneration and prevented mitochondrial swelling in paclitaxel treated mice treated and maintained the number of axonal mitochondria in paclitaxel-exposed cultured primary DRG neurons; and 4) GSSSG attenuated the increase in superoxide levels in primary cortical neurons incubated with paclitaxel. Taken together, these results suggest GSSSG ameliorates PIPN. The beneficial effects of GSSSG were associated with protection of mitochondrial integrity in peripheral nervous system axons.

As shown herein, oral administration of GSSSG at 50 mg/kg/day for 4 weeks ameliorated paclitaxel-induced mechanical allodynia and ³⁴S-labeled GSSSG was detected in lumber DRG and lumber spinal cord 2 hours after single oral administration. The results showed that GSSSG was well absorbed from the gastrointestinal tract and readily taken up by central and peripheral nervous tissues [33]. The concentrations of related polysulfides and persulfides containing ³⁴S were more than 10-fold greater than those of respective endogenous polysulfides and persulfides 2 hours after ³⁴S-labeled GSSSG administration. These observations suggest that GSSSG is in dynamic equilibrium with other reactive sulfur species. This study demonstrated an association between neuroprotective effects and increased levels of polysulfides in the peripheral nervous system after systemic administration of a polysulfide donor.

Impairment of mitochondria and increased oxidative stress play crucial roles in PIPN [34]. Mitochondria are a main source of cellular ROS but are also important targets of ROS. Normal mitochondria release low levels of ROS, as small amounts of electrons leak from complex I, III, and IV of the electron transport chain (ETC) and bind with molecular oxygen to produce superoxide. When electron transport is impaired, more electrons leak from ETC complexes to produce more ROS. Increased ROS production by dysfunctional mitochondria further impairs mitochondrial function, eventually leading to collapse of the mitochondrial membrane potential. Defective mitochondria are degraded and replaced by quality control mechanisms, such as mitophagy. Because neurons predominantly rely on oxidative phosphorylation in mitochondria to produce ATP, mitochondrial dysfunction causes degeneration of neuronal axons [35].

Reactive sulfur species are thought to protect mitochondria through several mechanisms: 1) supporting bioenergetics [36]; 2) scavenging ROS [14]; 3) activating superoxide dismutase (SOD) [37]; 3) preventing fission of mitochondria by inhibiting Drp1 activity [36]; and 4) upregulating the Nrf2/Keap1 pathway by persulfidation of Keap1 [38]. In the current study, administration of GSSSG attenuated paclitaxel-induced ROS production and prevented swelling and loss of mitochondria in peripheral neural axons. These observations suggest that GSSSG has anti-oxidative effects and that GSSSG may support bioenergetics. The present results in primary DRG neurons suggest that GSSSG prevents not only mitochondria fragmentation (or fission) induced by paclitaxel, but also loss of mitochondria in neural axons. As reported previously, redox balance and glutathione oxidation in neural axons partly regulate mitochondrial transportation from cell body to axon [39], and loss of axonal mitochondria results in axonal degeneration and neuropathy [41]. The present observations support that GSSSG attenuates PIPN via protecting mitochondria by regulating redox balance in peripheral neurons. Interestingly, paclitaxel, without or with GSSSG, upregulated Nrf2 a master regulator of cellular homeostasis, redox balance, and inflammation [42], suggesting that paclitaxel triggers an antioxidant defense mechanism. However, we observed that GSSSG with paclitaxel, but not paclitaxel alone, upregulated expression of HO1, NQO1 and GCLC, which are the downstream genes of Nrf2. Without wishing to be bound by theory, one possible reason why GSSSG upregulates genes that are downstream of Nrf2 is that GSSSG induces persulfidation of Keap1, a Nrf2 binding protein. Persulfidation of Keap1 promotes activation of Nrf2, thereby upregulating the downstream genes and exerting an antioxidative effect [38]. In addition to the Nrf2 pathway, GSSSG may exert protective effects on peripheral neurons via multiple antioxidant mechanisms.

### Methods of Treatment

As shown herein, systemic administration of GSSSG ameliorated paclitaxel-induced mechanical allodynia in a model of PIPN. After oral administration, GSSSG reached DRG and prevented impairment of mitochondria and axonal degeneration in peripheral neurons. GSSSG upregulated Nrf2-dependent antioxidant signaling pathway in vivo and diminished ROS levels in vitro. Because impairment of mitochondria is a common feature of diverse forms of peripheral neuropathy [42], these results imply potential therapeutic effects of GSSSG, not only for CIPN, but also for other forms of peripheral neuropathy caused by mitochondrial dysfunction, such as diabetic neuropathy.

Thus, provided herein are methods for the treatment, or reduction of risk, of disorders associated with peripheral neuropathy in a subject, e.g., a mammalian subject, e.g., a human or non-human veterinary subject. In some embodiments, the disorder is CIPN, e.g., PIPN. In some embodiments, the disorder is diabetic neuropathy, which has sensory nerve involvement and usually causes neuroaxonal damage in peripheral neurons, and is pathologically similar to CIPN. In some embodiments, the disorder is Guillan-Barré syndrome neuropathy, which has sensory nerve involvement and usually causes neuroaxonal damage in peripheral neurons, and is and is pathologically similar to CIPN. In some embodiments, the disorder is chronic inflammatory demyelinating polyneuropathy, which has similar unmyelinating as in CIPN. In some embodiments, the disorder is neuropathy caused by shingles (i.e., post herpetic neuralgia), which affects peripheral neurons. In some embodiments, the disorder is peripheral neuropathy caused by-hepatitis B, hepatitis C, myeloma, lymphoma, or kidney disease.

Generally, the methods include systemic (e.g., oral or nasal) administration of a therapeutically effective amount of a composition comprising a crystalline form of GSSSG, PTN-SSS, or LA-SSS as described herein, to a subject who is in need of, or who has been determined to be in need of, such treatment.

As used in this context, to “treat” means to ameliorate at least one symptom of the disorder associated with neurodegeneration. The conditions that can be treated using a method described herein can be associated with peripheral neuropathy and pain. Administration of a therapeutically effective amount of a compound described herein can result in reduced peripheral neuropathy and pain.

In addition, the methods can result in a reduction in risk of developing peripheral neuropathy and pain. Subjects who are at risk of developing peripheral neuropathy and pain can include those who have cancer and are being treated with a chemotherapy regimen including an agent that induces or can induce peripheral neuropathy and pain, e.g., a platinum-containing agent such as cisplatin, paclitaxel, vincristine, carboplatin, oxaliplatin, or nedaplatin. These methods can include oral or nasal administration an effective amount of a GSSSG, PTN-SSS, or LA-SSS composition as described herein within a few minutes to hours or days before and/or after administration of the chemotherapy. Subjects who are at risk of developing neuropathy and pain can include those who have (or have been diagnosed as having) diabetic neuropathy, Guillan-Barré syndrome neuropathy, chronic inflammatory demyelinating polyneuropathy, shingles, hepatitis B, hepatitis C, myeloma, lymphoma, or kidney disease.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. The compositions can be administered one from one or more times per day to one or more times per week; including once every other day. In some embodiments, the GSSSG, PTN-SSS, or LA-SSS is administered every day for at least 2, 3, 4, 5, 6, or 7 days before or after a dose of chemotherapy is administered, or every day for the first one or two weeks of a chemotherapy regimen comprising administration of a chemotherapy that can cause peripheral neuropathy. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity, and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Pharmaceutical Compositions and Methods of Administration

The methods described herein include the use of pharmaceutical compositions comprising GSSSG, PTN-SSS, or LA-SSS as an active ingredient. In some embodiments, the compositions are prepared using a crystalline form of GSSSG, using methods described in EP 3560947, by dissolving the crystalline GSSSG in a buffer, e.g., saline, at pH 3-6, e.g., pH 4.8-5. pH can be adjusted with acids or bases, such as hydrochloric acid or sodium hydroxide. Compositions comprising PTN-SSS or LA-SSS can be prepared by dissolving in a buffer, e.g., saline or water, at pH 4-9, e.g., 5-8.

An exemplary method for producing the crystal form of glutathione trisulfide dehydrate can comprise precipitating a crystal of glutathione trisulfide dihydrate in an aqueous solution in which glutathione trisulfide is dissolved, and collecting the precipitated crystal of glutathione trisulfide dihydrate. PTN-SSS or LA-SSS can be prepared as described in WO2022/045212 (LA-SSS) and WO2022/045052 (PTN-SSS).

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions for use in the present methods are formulated to be compatible with nasal or oral administration, or parenteral, e.g., intravenous, administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY).

Oral compositions generally include an inert diluent or an edible carrier. For the purpose of oral therapeutic administration, the active agents can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or agents of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or fruit (e.g., orange or cherry) flavoring.

For the purpose of mucosal therapeutic administration, the active compound (e.g., GSSSG, PTN-SSS, or LA-SSS) can be incorporated with excipients or carriers suitable for administration by inhalation or absorption, e.g., via nasal sprays or drops. For nasal administration, the formulations can be, for example, an aerosol or dry powder in a sealed vial or other suitable container.

The pharmaceutical compositions and dosage forms can further comprise one or more compounds that reduce the rate by which an active ingredient will decompose. Thus, the oral or nasal dosage forms described herein can be processed into an immediate release or a sustained release dosage form. Immediate release dosage forms may release the GSSSG, PTN-SSS, or LA-SSS in a fairly short time, for example, within a few minutes to within a few hours. Sustained release dosage forms may release the GSSSG, PTN-SSS, or LA-SSS over a period of several hours, for example, up to 24 hours or longer, if desired. In either case, the delivery can be controlled to be substantially at a certain predetermined rate over the period of delivery.

Nasal delivery is considered an attractive route for needle-free, systemic drug delivery, especially when rapid absorption and effect are desired. In addition, nasal delivery may help address issues related to poor bioavailability, slow absorption, drug degradation, and adverse events (AEs) in the gastrointestinal tract and avoids the first-pass metabolism in the liver.

Liquid nasal formulations are mainly aqueous solutions, but suspensions and emulsions can also be delivered. In traditional spray pump systems, antimicrobial preservatives are typically required to maintain microbiological stability in liquid formulations.

Metered spray pumps can be used. The pumps typically deliver about 25-200 μL per spray, and they offer high reproducibility of the emitted dose and plume geometry. The particle size and plume geometry can vary within certain limits and depend on the properties of the pump, the formulation, the orifice of the actuator, and the force applied. Traditional spray pumps replace the emitted liquid with air, and preservatives are therefore required to prevent contamination.

Alternative spray systems or devices that avoid the need for preservatives can also be used. These systems use a collapsible bag, a movable piston, or a compressed gas to compensate for the emitted liquid volume. The solutions with a collapsible bag and a movable piston compensating for the emitted liquid volume offer the additional advantage that they can be emitted upside down, without the risk of sucking air into the dip tube and compromising the subsequent spray. This may be useful for some products where the patients are bedridden and where a head down application is recommended. Another method used for avoiding preservatives is that the air that replaces the emitted liquid is filtered through an aseptic air filter. In addition, some systems have a ball valve at the tip to prevent contamination of the liquid inside the applicator tip.

For administration by inhalation, the GSSSG, PTN-SSS, or LA-SSS compounds can be delivered in the form of a dry powder or an aerosol spray from pressured container or dispenser that contains a suitable propellant, e.g., a gas such as carbon dioxide, or a nebulizer. Such methods include those described in U.S. Pat. No. 6,468,798.

Devices for nasal administration, e.g., as described herein, comprising GSSSG, PTN-SSS, or LA-SSS are also provided herein.

Described herein are kits that can include a composition comprising GSSSG, PTN-SSS, or LA-SSS, e.g., as an already prepared dry powder or liquid nasal or oral form ready for administration or, alternatively, can include a composition comprising GSSSG, PTN-SSS, or LA-SSS as a solid pharmaceutical composition that can be reconstituted with a solvent to provide a liquid nasal or oral dosage form. When the kit includes GSSSG composition as a solid pharmaceutical composition that can be reconstituted with a solvent to provide a liquid dosage form (e.g., for oral or nasal administration), the kit may optionally include a reconstituting solvent at pH 3-6, e.g., pH 4.8-5.0. When the kit includes PTN-SSS, or LA-SSS composition as a solid pharmaceutical composition that can be reconstituted with a solvent to provide a liquid dosage form (e.g., for oral or nasal administration), the kit may optionally include a reconstituting solvent at pH 4-9, e.g., pH 5-8. In this case, the constituting or reconstituting solvent is combined with the active ingredient to provide a liquid oral dosage form of the active ingredient. Typically, the active ingredient is soluble in the solvent and forms a solution. The solvent can be, e.g., water, a non-aqueous liquid, or a combination of a non-aqueous component and an aqueous component. Suitable non-aqueous components include, but are not limited to oils; alcohols, such as ethanol; glycerin; and glycols, such as polyethylene glycol and propylene glycol. In some embodiments, the solvent is phosphate buffered saline (PBS).

The pharmaceutical compositions can be included in a container, pack, or dispenser together with instructions for administration. For example, the GSSSG can be provided in a kit in a crystalline form with a sterile buffer (e.g., saline) at pH 3-6 for use in dissolving the crystals to prepare a solution for nasal or oral administration.

### EXAMPLES

The invention is further described in the following examples, which do not limit the scope of the invention described in the claims.

### Materials and Methods

The following materials and methods were used in the Examples below.

### Animals

All animal protocols were approved by the Massachusetts General Hospital Institutional Animal Care and Use Committee. Animals were cared for in accordance with the guidelines established by the NIH and the International Association for the Study of Pain [16]. Male C57BL/6J mice (6-7 weeks old) were purchased from the Jackson Laboratory (Bar Harbor, ME, USA). The mice were housed in a 12-hour shift light-control environment from 7 am to 7 μm with ad libitum access to food and water in our animal facility until the time of experiments.

### Drugs and Animal Models

Paclitaxel (Sigma-Aldrich) was dissolved with ethanol and cremophor (1:1) and diluted with normal saline (1:4). Peripheral neuropathy was induced in mice by intraperitoneal (i.p.) administration of 4 mg/kg paclitaxel every other day for a total of 4 injections (cumulative dose of 16 mg/kg), according to a previously described protocol [17]. A stable, crystallized form of GSSSG was produced and provided by Kyowa Hakko Bio CO., LTD. GSSSG was pulverized and mixed in 0.5% methylcellulose. To evaluate the therapeutic effects of GSSSG in a model of PIPN, mice were randomly divided into three groups of 6. Mice were treated with: 1) paclitaxel alone; 2) paclitaxel and GSSSG; 3) or vehicle alone (control). Mice in groups 1 and 2 received paclitaxel as described above, while mice in the control group received the same volume of vehicle. The first dose of GSSSG was given within 1 hour after the first PTX injection. Treatment was administered as oral gavage for 28 days with GSSSG at 50 mg/kg/day (group 2) or 0.5% methylcellulose (groups 1 and 3). The dose of GSSSG was determined based on pilot studies. To determine the effect of GSSSG on the development of PIPN, behavioral tests to assess allodynia and hyperalgesia were conducted on all mice on days-1 (baseline), 7, 14, 21, and 28 after paclitaxel administration. Behavioral tests were performed by an investigator who was blinded to the treatment groups, as described in the next section.

PTN-SSS was synthesized and provided by Kyowa Hakko Bio Co., Ltd. The purity of PTN-SSS is 96.3%, and PTN-SSS is highly water soluble (>50 g/L). PTN-SSS and PTN (Toronto Research Chemicals, Toronto, ON, Canada) were suspended in distilled water. PTN-SSS is stable in solution at pH 4.0-9.0 at room temperature for at least 4 days. In the PTN-SSS study, distilled water was used as the vehicle alone.

To determine the tissue distribution of orally-administered GSSSG, ³⁴S-labelled GSSSG was administered by gavage to each of four mice. ³⁴S-labelled GSSSG was synthesized and provided by Kyowa Hakko Bio CO., LTD. Two hours after a single dose of ³⁴S-GSSSG, DRG, lumber spinal cord, brain, liver, and plasma were obtained and snap frozen for later analysis.

### Behavior Test

### 1) Measurement of Mechanical Allodynia

Mechanical withdrawal thresholds were measured by the manual von Frey filament test to assess mechanical allodynia. Before testing, mice were habituated in our animal facility for 3-4 days and acclimated in a plastic cage on a wire mesh floor for 30 minutes for 3 consecutive days. In the test, mice were placed in the same cage as in the acclimation. An experimenter who was blinded to the treatment of mice stimulated the mid-plantar surface of their hind paws with von Frey filaments (Ugo Basile, Cat No. 37450-275) in the range from 0.04 gram force (g) to 2.0 g, starting from 0.6 g. Filaments were applied with constant speed until they bent. Behavioral responses, withdrawal and licking of their paws, were considered reactive. The stimulation of the same size filament was repeated up to 2 times if the mouse did not react. The filament was changed according to the previous filament response (we applied a smaller filament after positive response and a larger filament after negative response). The total applied number of filaments was up to 9 times. The 50% threshold of a paw withdrawal response was calculated by the Up-down Reader [18] based on the up and down method [19]. This behavior test was conducted weekly for 4 weeks.

### 2) Hot Plate Test

To assess hyperalgesia, the heat threshold was determined by the hot plate test [20]. Mice were placed on the hot plate (Hot/Cold Plate NG, Ugo Basile, Cat No. 35150) warmed at 52° C. The latency response time was measured until they showed nociceptive behavior, including hind paw withdrawal or licking, stamping, and jumping. The test was repeated 2 times after 5 minutes interval by a blinded examiner, and the average time was calculated. The ratio of change from the baseline was calculated from the difference of the latency response time from the baseline divided by the baseline time. This test was conducted weekly in the same group of mice after the von Frey test for 4 weeks.

### Immunohistochemistry Staining of Intraepidermal Nerve Fibers (IENF)

Density of intraepidermal nerve fiber (fibers/mm) of hind paws was calculated to evaluate peripheral nerve fiber damage by paclitaxel. Mice were deeply anesthetized with Isoflurane (4%) and euthanized by exsanguination on week 1 or week 4 after paclitaxel treatment for tissue harvest. They were perfused with cold 4% paraformaldehyde in PBS through the left ventricle. Skin from their hind paws was harvested and fixed in 4% paraformaldehyde overnight, then cryoprotected in 20% sucrose solution at 4° C. until they sank and in 30% sucrose solution overnight at 4° C. Tissue blocks were then submerged in optimal cutting temperature medium, frozen at −80° C., and sliced in a cryostat (25 μm). The sections were then incubated with 0.3% hydrogen peroxide for 10 minutes and with blocking solution (5% Donkey serum; Sigma-Aldrich, Cat No. D9663, 0.3% Triton-114) for one hour. Subsequently, sections were incubated overnight at 4° C. with anti-protein gene product (PGP) 9.5 antibody (1:100, rabbit, Abcam, Cat No. ab108986) and anti-collagen IV antibody (1:400, goat, SouthernBiotech, Cat No. 1340-01). They were incubated with the secondary antibodies (1:300, donkey anti-rabbit, Abcam, Cat No. ab150073; donkey anti-goat, Abcam, Cat No. ab150132) for one hour at room temperature and covered with fluoroshield mounting medium with DAPI (Sigma-Aldrich, Cat No. F6057). Ten images per mouse (6 mice per group) were taken by fluorescence microscopy (Nikon Eclipse 80i, Nikon Instruments, Inc., Melville, NY), and four out of ten images were randomly selected. The density (fibers/mm basement membrane) was calculated as the number of intraepidermal nerve fiber divided by the length of basement membrane in accordance with the guideline [21].

### Histological Evaluation of Sciatic Nerves

### 1) Toluidine Blue Staining

Four weeks after paclitaxel treatment, sciatic nerves were evaluated by toluidine blue staining as described previously [22]. Briefly, mice were deeply anesthetized, and their sciatic nerves were exposed in the prone position. Sciatic nerve was covered with Trump's fixative (Quimigen, Cat No. 11750) for 10 minutes. Subsequently, it was harvested and fixed in the same fixative for one week by changing the fixative every other day. Samples were immersed in 2% osmium tetroxide (TGI, Cat No. O0308) for 2 hours and embedded in Resin-Epoxy medium (Sigma-Aldrich, Cat No. 45359-4) overnight at 60° C. by following the protocol provided by the manufacturer. The embedded nerve block was sectioned at 1 μm by an ultramicrotome (Reichert-Jung, Ultracut E, Austria) and stained in 1% toluidine blue (Sigma-Aldrich, Cat No. T3260). Two images per mouse (6 mice per group) were taken by light microscopy (Nikon Eclipse 80i, Nikon Instruments, Inc., Melville, NY) and analyzed by ImageJ. Thickness of myeline was evaluated by G-ratio, a ratio of inner radius over outer radius. G-ratio was calculated using GRatio software, an ImageJ plugin available online (http://gratio.efil.de/) that converts inner and outer perimeter of myelin to the radius. Counting neurons and measuring the size were conducted by an examiner who was blinded to the treatment of mice.

### 2) Transmission Electron Microscopy

To evaluate unmyelinated neurons and mitochondria, we examined neurons in sciatic nerve by a transmission electron microscope. Resin-embedded nerve blocks using the same method above were sectioned at 50 nm by an ultramicrotome. Sections were examined with an FEI Morgagni transmission electron microscope. Low (×2200) and high (×11000) magnification of images were captured with an AMT 2K charge-coupled device camera (Advanced Microscopy Techniques, Woburn, MA, USA). Unmyelinated neurons were quantified in 9 low magnified images per group (3 mice per group). The percentage of unmyelinated neurons was calculated by dividing unmyelinated neurons by the total number of neurons. The cross-sectional area of mitochondria (μm²) in unmyelinated neurons was quantified in 18 to 20 high magnified images per group (3 mice per group). In total, 80 to 100 mitochondrion per group were evaluated. Quantification was conducted by a blinded examiner, and the images were evaluated by a pathologist (A S-R) blinded to the identity of samples.

Real-Time Quantitative Polymerase Chain Reaction (qPCR)

Changes in gene expression in lumber dorsal root ganglions (DRG) induced by paclitaxel and GSSSG were examined with real-time qPCR after a single administration of paclitaxel with or without GSSSG. We simultaneously administered 16 mg/kg paclitaxel by intraperitoneal injection and 50 mg/kg GSSSG by oral gavage and examined the mRNA expression 2 hours later. Lumbar DRG were isolated from mice as described previously [23]. Removed DRG were immersed in RNA later (Invitrogen, Cat No. AM7020) at 4° C. overnight. After retrieving from RNA later, they were stored at −80° C. DRGs were homogenized in TRIzol reagent (ThermoFisher Scientific, Cat No. 15596026) and mixed with chloroform. After centrifugation at 14000×g at 4° C. for 15 minutes, the transparent top layer was transferred to a new tube. The samples were mixed with 400 μl of isopropanol and incubated at −20° C. for 20 minutes. After centrifugation at 14000×g at 4° C. for 15 minutes, the pellets were collected, mixed with 70% ethanol, and centrifuged at 14000×g at 4° C. for 10 minutes. The pellets were dried for 10 minutes and incubated with 50 μl of nuclease-free water. Complementary DNA was synthesized using the cDNA Reverse Transcription Kit (Applied Biosystems, Cat No. 4368814) and quantitative PCR was performed by using SYBR green (Applied Biosystems, Cat No. A46109). Primers are listed in Table 1. Relative quantification of gene expression was performed via 2^{-ΔΔCT} method.

TABLE 1
List of primer sequences for quantitative
polymerase chain reaction.

| | | | SEQ |
|---|---|---|---|
| | | | ID |
| Gene | F/R | Sequence | NO: |
| Nrf2 | Forward | 5′-TCCTCAGCAGAACAGGAACAG-3′ | 1 |
| | Reverse | 5′-CCTCCAAAGGATGTCAATCAA-3′ | 2 |
| HO1 | Forward | 5′-AAGCCGAGAATGCTGAGTTC-3′ | 3 |
| | Reverse | 5′-GCCGTGTAGATATGGTACAAGGA-3′ | 4 |
| NQ01 | Forward | 5′-AGGATGGGAGGTACTCGAATC-3′ | 5 |
| | Reverse | 5′-AGGCGTCCTTCCTTATATGCTA-3′ | 6 |
| GCLC | Forward | 5′-GGACAAACCCCAACCATCC-3′ | 7 |
| | Reverse | 5′-GTTGAACTCAGACATCGTTCCTC-3′ | 8 |
| 18S | Forward | 5′-CGGCTACCACATCCAAGGAA-3′ | 9 |
| | Reverse | 5′-GCTGGAATTACCGCGGCT-3′ | 10 |

Abbreviations:
GCLC, Glutamate-Cysteine Ligase Catalytic Subunit; HO1, Heme Oxygenase 1; NOQ1, NAD(P)H Quinone Dehydrogenase 1; Nrf2, Nuclear factor-erythroid factor 2-related factor 2.

### Mass Spectrometry to Detect GSSSSG Administered by Oral Gavage

We examined whether administrated GSSSG via oral gavage reaches peripheral nervous system using liquid chromatography with tandem mass spectrometry (LC-MS/MS). Fifty mg/kg of ³⁴S-labeled GSSSG (in which the middle sulfur, that is a sulfane sulfur, was replaced to ³⁴S; G-³²S-³⁴S-³²S-G) was orally administered to mice. Two hours after administration, plasma and tissue from liver, brain, lumbar spinal cord, and lumbar DRG (L1 to L6) were collected and immediately frozen at −80° C. Tissues were homogenized with 5 mM of β-(4-hydroxyphenyl)ethyl iodoacetamide (HPE-IAM) (Santa Cruz, Cat No. SC-473766) and incubated for 20 minutes at 37° C. to promote HPE-IAM reaction that stabilizes persulfide residues [24]. Proteins were removed by centrifugation at 15,000 g for 10 minutes at 4° C., and total protein concentration was measured by BCA assay. The supernatant was diluted by 0.1% formic acid for LC-MS/MS analysis. The amount of ³⁴S-labeled GSSSG was quantified in selective reaction monitoring (SRM) with precursor ion (647.14 m/z), product ion (389.1 m/z), and HCD (21 v) and normalized by protein concentration. The ratio of ³⁴S-labeled reactive sulfur species to endogenous (³²S) reactive sulfur species (GSSH, CysSSH, and CysSSSCys) were also calculated from their peak area measured by Dionex UltiMate 3000 RS UPLC-Orbitrap Exploris 480 mass spectrometer (Thermo Scientific Scientific, Waltham, MA, USA). In brief, samples were subjected to the UPLC system with a Hypersil Gold C-18 (100×2.1 mm, 3.0 μm, Thermo Fisher Scientific) column and they were eluted by using a linear methanol gradient of the mobile phase (0-90%, 15 minutes) in the presence of 0.1% formic acid at a flow rate of 0.2 ml/minutes at 40 degrees. The raw data were obtained by Compound Discoverer software 3.3. Molecular weight of reactive sulfur species combined with HPE-IAM were reported in previous studies [25, 26].

### In Vitro Studies

### 1) Primary DRG Neuron Isolation and Histological Evaluation

Primary DRG neurons were prepared from 8 to 10-week old mice as described previously [27]. Briefly, lumber DRG (L1 to L6) were isolated from mice and centrifuged at 176×g (1000 rpm) for 3 minutes. After aspirating the solution, DRG were incubated with Dispase-II Solution (Sigma-Aldrich, Cat No. SCM133) and collagenase type II (Worthington, Cat No. LS004176) for 70 minutes and with 0.25% trypsin for 5 minutes. Subsequently triturated by frame polished glass pipet, neurons were seeded on a 12-well plate with 15 mm coverslip. Cells were incubated in Neurobasal A-medium with 2% B27 supplement (Gibco, Cat No. 10889038), 1% penicillin/streptomycin, 1% Glutamax (Gibco, Cat No. 35050061), and nerve growth factor (Sigma Aldrich, Cat No. N6009) for 24 hours. After 24 hours, they were exposed to 100 nM of paclitaxel with or without 500 nM of GSSSG or PTNSSS for one hour and 50 nM of MitoTracker (Invitrogen, Cat No. M22426) for 30 minutes. After fixing by 4% paraformaldehyde for 10 minutes at room temperature, cells were immersed in 0.2% Triton X-100 in PBS for 7 minutes and washed by PBS. They were incubated with anti-NF200 antibody (1:400, mouse, Sigma-Aldrich, Cat No. N0142) overnight at 4° C. and with the secondary antibody (1:1000, donkey anti-mouse, Abcam, Cat No. ab150105) for one hour at room temperature. After being covered with fluoroshield mounting medium with DAPI (Sigma-Aldrich, Cat No. F6057), 9-10 cells per group (3 mice per group) were imaged on confocal microscopy (ZEISS LSM 800, Carl Zeiss, Thornwood, NY) using a 63× oil immersion objectives lens with 1,024×1,024 pixels image size.

### 2) Primary Cortical Neuron Isolation and ROS Assay

Primary cortical neurons were prepared from the cortex of mice on embryonic day 15, as described previously [28]. In brief, the embryo's cortex was isolated in Hanks' balanced salt solution and centrifuged at 176×g (1000 rpm) for 3 minutes. After aspirating the solution, cells were incubated with 0.25% trypsin for 15 minutes and seeded 20,000/well in a 96-well plate coated with the poly-D-lysin (Gibco, Cat No. A3890401). They were incubated in Neurobasal medium (Gibco, Cat No. 21103049) with 2% B27 supplement (Gibco, Cat No. 10889038), 1% penicillin/streptomycin, and 1% Glutamax (Gibco, Cat No. 35050061) until day 11 when they were used for experiments.

To examine whether GSSSG reduces ROS generated by paclitaxel, we conducted Dihydroethidium (DHE) assay (Abcam, Cat No. ab236206) that is sensitive to superoxide. The experiment was done according to protocol provided by the manufacturer. Briefly, primary cortical neurons were incubated in DHE reagent with or without 10 and 30 μM of GSSSG for 30 minutes. After incubating, 100 nM of paclitaxel was administered, and cells were incubated for one hour. Fluorescence of DHE was measured at 490 nm of excitation and 585 nm of emission wavelength.

### 3) Cancer Cell Line and Viability

To examine whether co-administration of GSSSG affects anti-tumor effects of paclitaxel, we conducted experiments using MDA-MB-231, a human breast cancer cell line (HTB-26, ATCC). Cells were cultured in the medium of 90% RPMI1640 (Corning, Cat No. 10-040-CV), 10% FBS, and 1% penicillin/streptomycin. After being seeded into a 96-well plate at the density of 20,000/well and cultured overnight, they were exposed to different concentrations of paclitaxel (0.125 μM, 0.25 μM, 0.5 μM, 1 μM, and 2 μM) and incubated for 24 hours. Cell viability was evaluated by LDH Cytotoxicity Detection Kit (Roche, Cat No. 11644793001). Briefly, a plate was centrifuged at 250×g for 10 minutes. Cells were washed with PBS and incubated with 100 μl of 1% Triton X-100 at 37° C. for 30 minutes. After mixing with assay enzyme for 30 minutes at 25° C., absorbance was measured at wavelength 492 nm to determine cell viability. Inhibitory effects of paclitaxel were calculated by half-maximal inhibitory concentration (IC50). We utilized the IC50 of paclitaxel obtained with LDH assay to examine the anti-tumor effect of paclitaxel and co-administration of GSSSG using the trypan blue exclusion assay. We tested the effects of 10 μM of GSSSG because this concentration prevented axonal degeneration in primary cortical neurons. MDA-MB-231 cells were seeded 5.6×10,000/well in a 6-well plate and incubated overnight. The concentration of paclitaxel based on IC50 and 10 UM of GSSSG were applied and incubated for 24 hours. After being stripped by 0.25% trypsin, live cells were counted under 0.04% trypan blue. Five wells were evaluated per group.

### Statistical Analysis

Sample sizes for behavior test were chosen based on a previous study [29]. All values are expressed as mean±standard deviation (SD). The behavior test results were analyzed in the mixed effect model because the behavior was measured repeatedly over five times from the same mice, and individual differences at baseline were confirmed in the preliminary study. Bonferroni correction was applied to correct for multiple comparisons in the mixed effect model. Parametric data were analyzed by one-way analysis of variance (ANOVA) with Dunnett's multiple comparisons test. Non-parametric data were analyzed by Kruskal-Wallis test with Dunn's multiple comparisons test. Morphology in primary DRG neurons was analyzed by two-way repeated measures ANOVA with Dunnett's multiple comparisons test. Anti-tumor effect of paclitaxel with GSSSG was analyzed by equivalence test using two one-sided t-tests. The margin of equivalence was defined as 10% difference in cell count. Probability (p) value less than 0.05 was considered significant. Statistical analyses were performed using GraphPad Prism 9.1 (GraphPad Software Inc., La Jolla, CA, USA).

### Example 1. GSSSG Prevented Mechanical Allodynia Induced by Paclitaxel

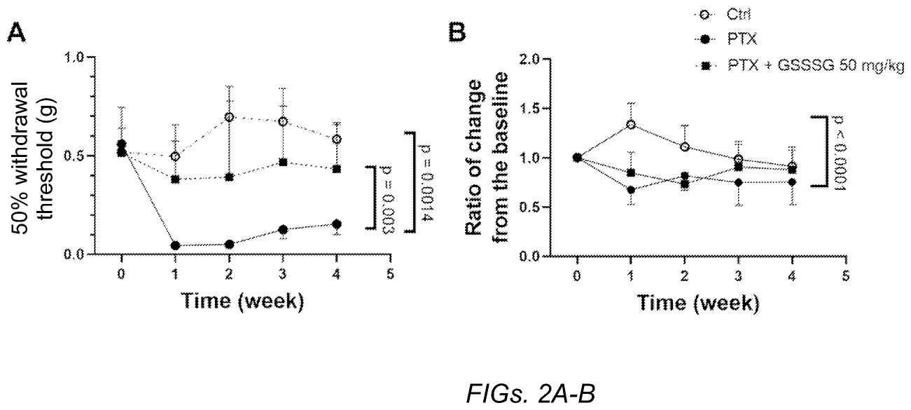

Male adult mice were treated with 4 mg/kg paclitaxel every other day for 4 injections (on days 0, 2, 4, and 6). From week 1 through week 4 after starting paclitaxel, paclitaxel treated mice showed a sign of mechanical allodynia by exhibiting significantly lower paw withdrawal threshold than control mice measured by the von Frey test (FIG. 2A closed circle, p=0.0014). Paclitaxel also induced thermal hyperalgesia measured by the hot plate test (FIG. 2B, closed circle, p<0.0001). Oral administration of GSSSG at 50 mg/kg/day mitigated mechanical allodynia over the experimental period (FIG. 2A, black square, p=0.003). On the other hand, threshold for thermal hyperalgesia was not altered by administration of GSSSG (FIG. 2B, black square).

### Example 2. GSSSG Prevented Loss of Intraepidermal Nerve Fibers Induced by Paclitaxel

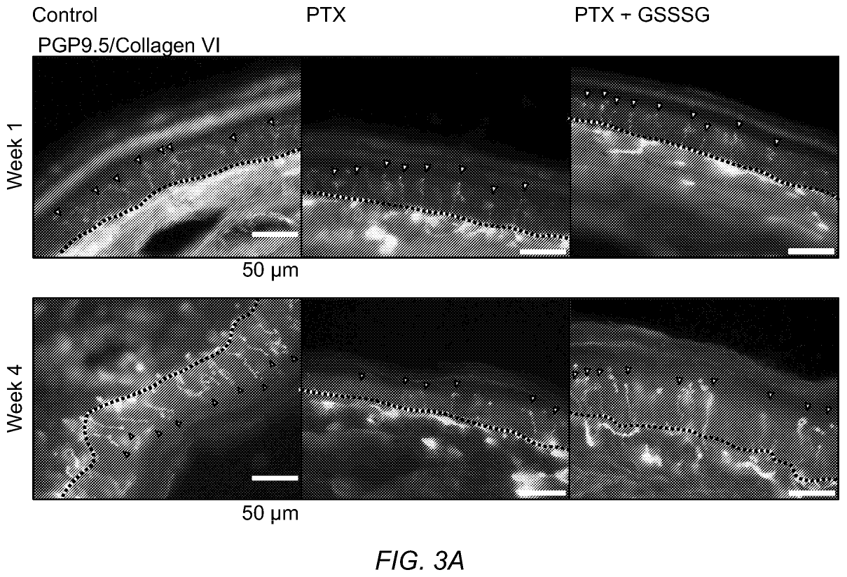

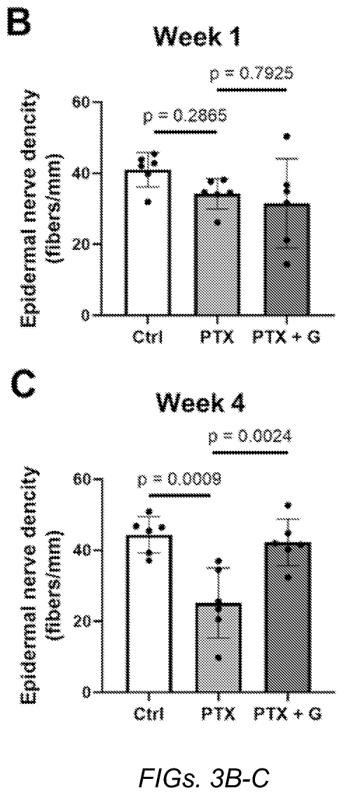

We evaluated degeneration of unmyelinated nerve endings by the density of intraepidermal nerve fibers, a widely used pathological indicator of peripheral neuropathy. The density of the intraepidermal nerve fibers in the planter surface skin of hind paws of mice was calculated by the number of intraepidermal nerve fibers (FIG. 3A, yellow arrowhead) divided by the length of epidermal basement membrane (FIG. 3A, white dashed line). Paclitaxel decreased the density of intraepidermal nerve fibers at 4 weeks, but not at 1 week, after starting paclitaxel (FIG. 3B, C). Daily oral administration of 50 mg/kg GSSSG prevented paclitaxel-induced loss of intraepidermal nerve fibers at 4 weeks after starting paclitaxel (p=0.0024).

### Example 3. GSSSG Prevented Loss of Unmyelinated Axons in the Sciatic Nerve Induced by Paclitaxel

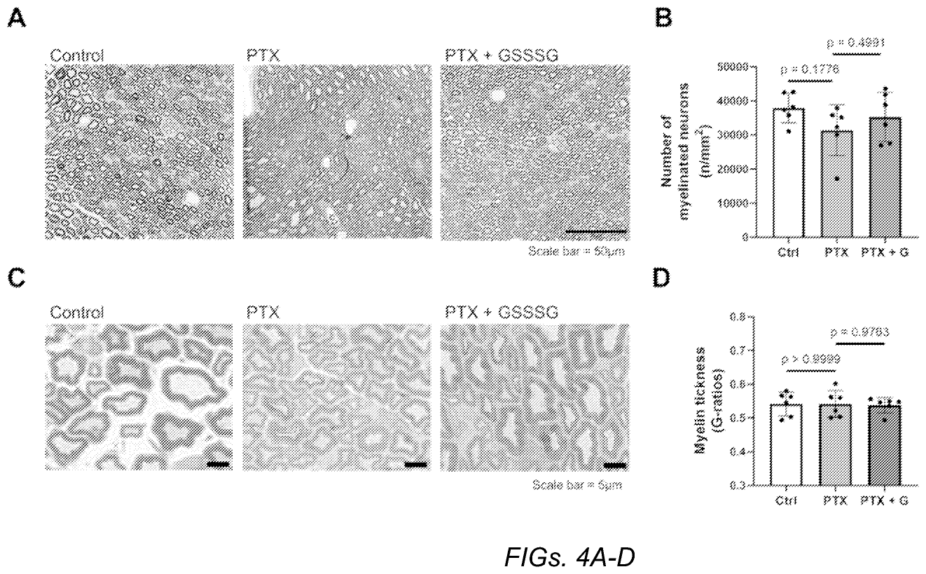

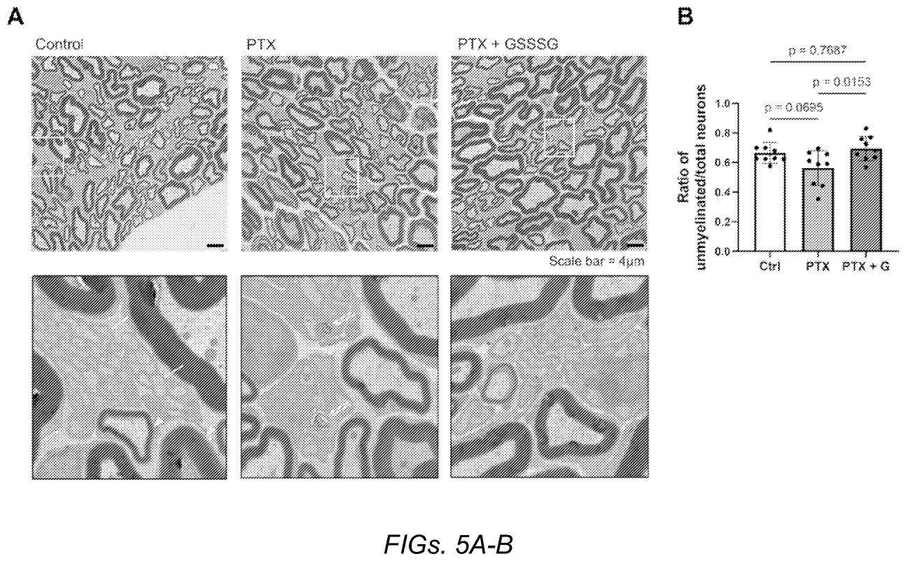

To evaluate the impact of paclitaxel on myelinated and unmyelinated neurons, we counted the number of neurons in the sciatic nerve at 4 weeks after starting paclitaxel. The number of myelinated neurons was similar between control, paclitaxel, and paclitaxel with GSSSG groups (FIG. 4A, B). Thickness of myeline calculated by G-ratio (a ratio of inner radius over outer radius) was not affected by paclitaxel without or with GSSSG (FIG. 4C, D). We also analyzed transmission electron microscopy images (low magnification, ×2200) to examine the number of unmyelinated neurons in the sciatic nerve. Unmyelinated (FIG. 5A, arrow) and myelinated neurons (FIG. 5A, arrowhead) were counted and divided by a total number of neurons. Paclitaxel tended to decrease unmyelinated neurons compared to control mice (p=0.0695) and made neurons out of alignment (FIG. 5A, double arrow), indicating axonal degeneration. Compared to mice that received paclitaxel alone, mice that received paclitaxel and GSSSG had a larger ratio of unmyelinated neurons in the sciatic nerve that was similar to observed in control mice (FIG. 5A, B, p=0.0153). These results suggest that GSSSG prevents axonal loss of unmyelinated neurons after paclitaxel treatment.

### Example 4. GSSSG Prevented Mitochondrial Swelling in Axons of Unmyelinated Neurons

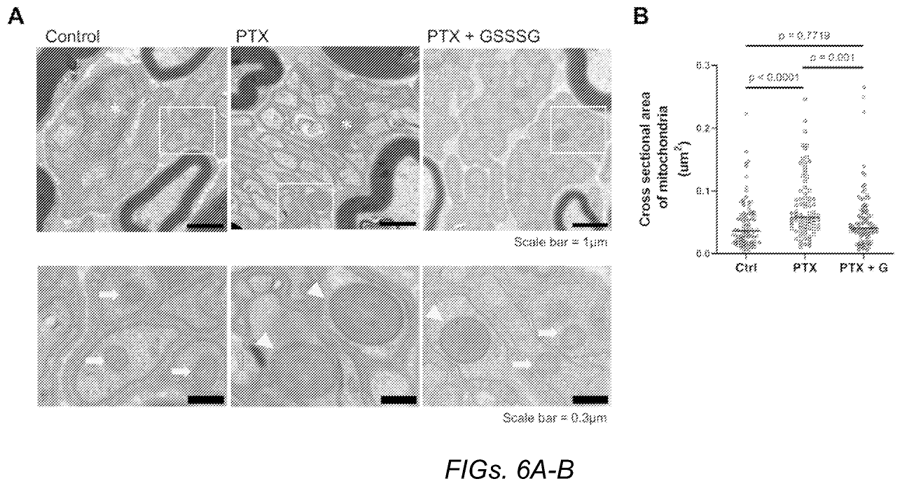

To explore mechanisms responsible for axonal degeneration of unmyelinated neurons by paclitaxel, we examined mitochondrial morphology in unmyelinated sciatic neurons 4 weeks after starting paclitaxel. Mitochondria appeared to be larger and swollen in unmyelinated sciatic neurons of paclitaxel-treated mice (FIG. 6A, arrowhead) compared to control mice and mice treated with paclitaxel and GSSSG. The cross-sectional area of mitochondria in unmyelinated sciatic neurons in paclitaxel-treated mice was significantly larger than those in control mice (FIG. 6B, p<0.0001) and mice treated with paclitaxel and GSSSG (FIG. 6B, p=0.001). These results implicate that beneficial effects of GSSSG against paclitaxel-induced axonal degeneration are mediated by protection of mitochondrial integrity.

### Example 5. ³⁴S-Labeled GSSSG was Detected in DRG, Spinal Cord, Brain, and Liver after Oral Administration

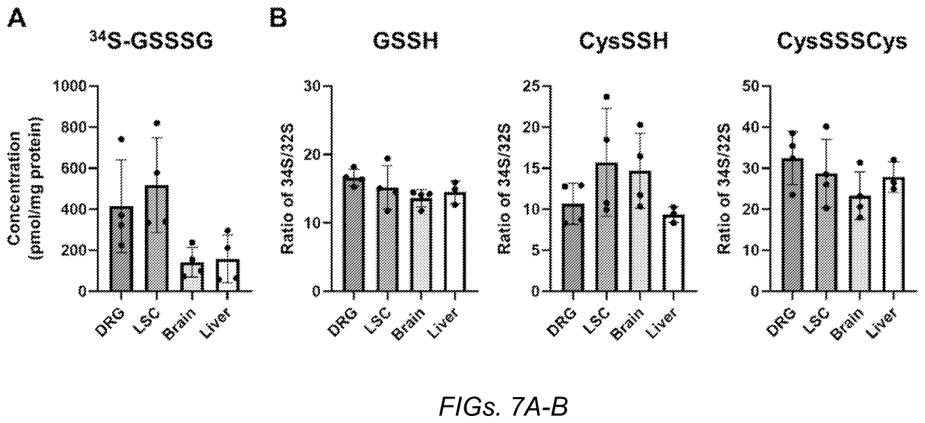

To study pharmacokinetics of orally administrated GSSSG, we examined distribution of GSSSG and its metabolites in central and peripheral nervous systems using ³⁴S-labelled GSSSG. Lumber DRG, lumber spinal cord, brain, liver, and plasma were harvested 2 hours after oral administration of ³⁴S-labelled GSSSG at 50 mg/kg, and levels of ³⁴S-labelled GSSSG in each tissue were determined by liquid chromatography-tandem mass spectrometry (LC-MS/MS). While endogenous GSSSG was not detected, the average concentration of administrated ³⁴S-labelled GSSSG was 415, 518, 142, and 158 pmol/mg protein in lumber DRG, lumber spinal cord, brain, and liver, respectively (FIG. 7A). Plasma level of GSSSG was 58 pmol/ml. We also determined the ratio of exogenous (containing ³⁴S) to endogenous (containing ³²S) reactive sulfur species: GSSH, CysSSH, and CysSSSCys, in these 4 tissues (FIG. 7B, C, D, respectively). Concentration of ³⁴S-labelled GSSH, CysSSH, and CysSSSCys was more than 10-fold higher than endogenous levels of GSSH, CysSSH, and CysSSSCys in all 4 tissues. These observations indicate that orally administered GSSSG was absorbed and uptaken into central and peripheral nervous systems and partially metabolized to other reactive sulfur species.

### Example 6. GSSSG and PTNSSS Prevented Paclitaxel-Induced Axonal Degeneration and Fragmentation of Mitochondria in Cultured Primary DRG Neurons

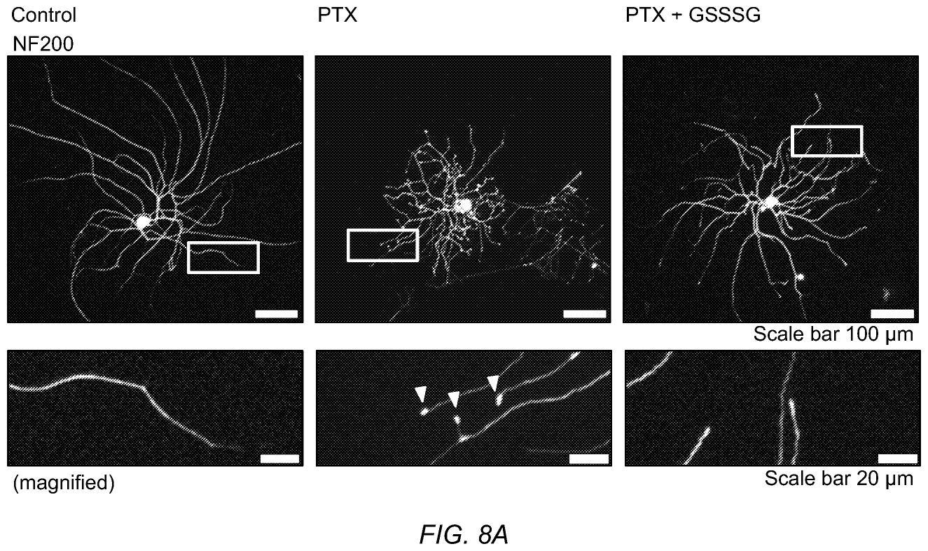

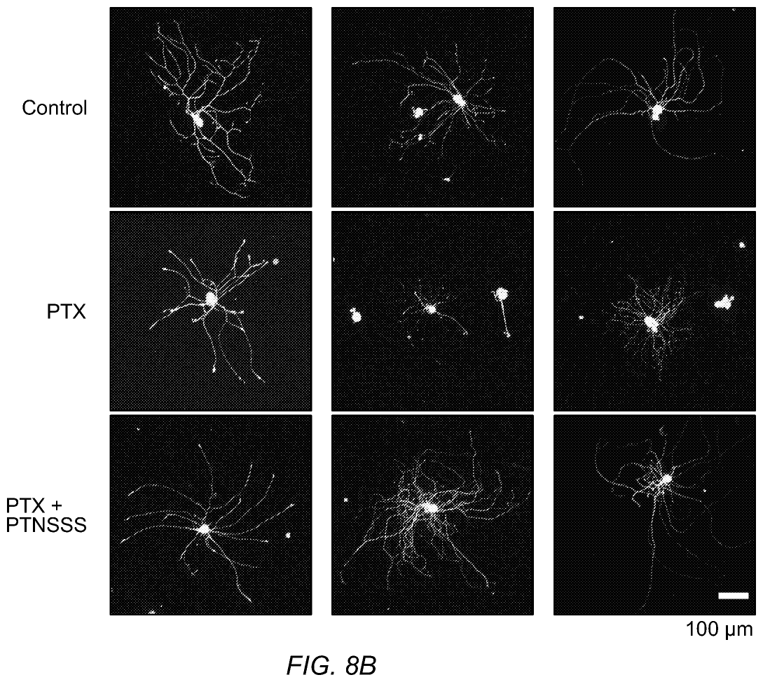

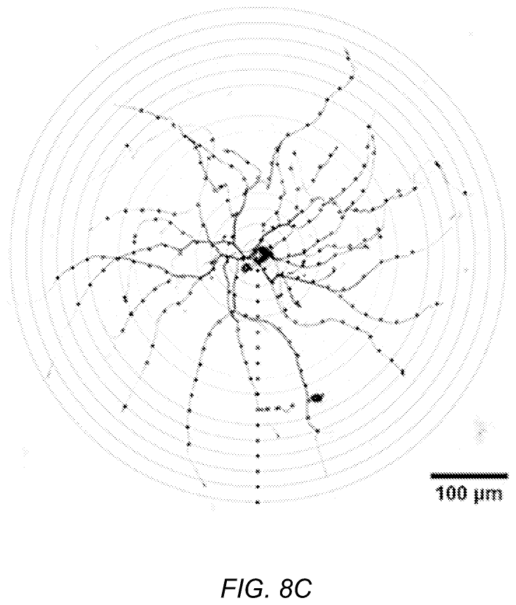

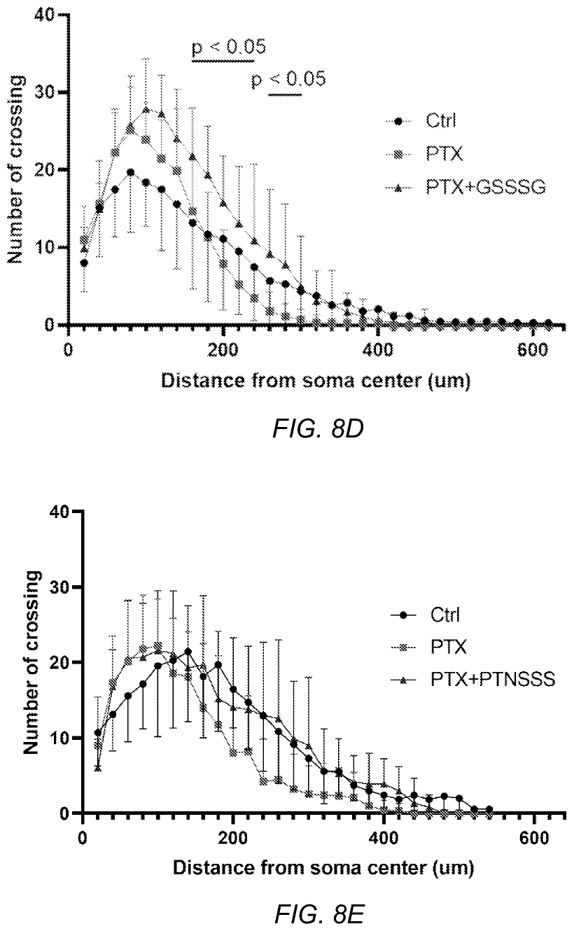

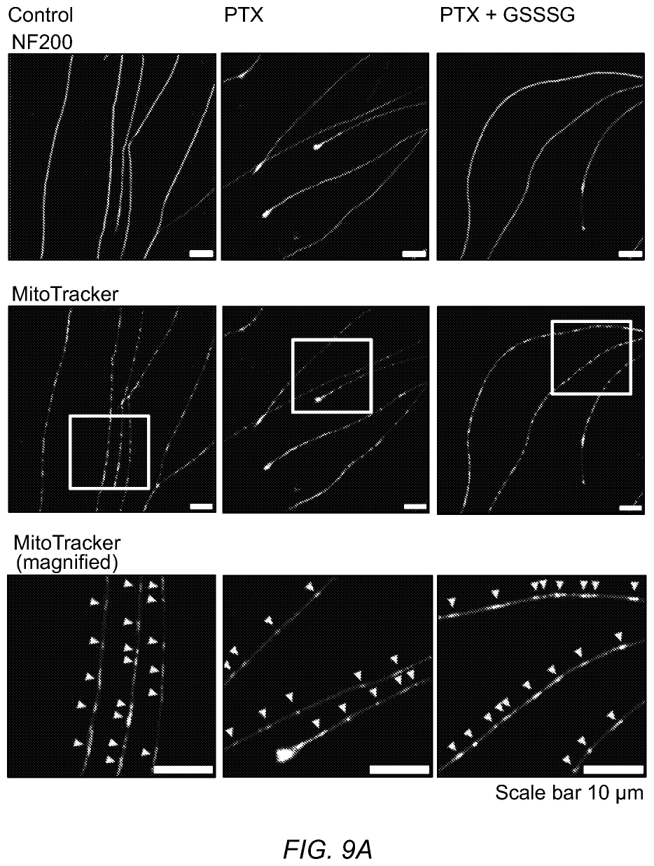

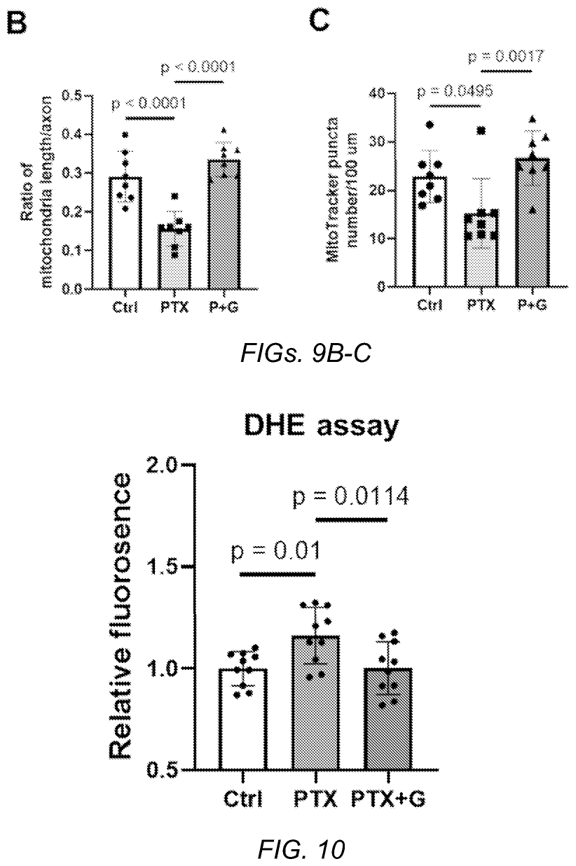

Effects of PTNSSS and GSSSG on axonal integrity were examined in cultured murine primary DRG neurons incubated with paclitaxel. Neurons treated with 100 nM of paclitaxel for one hour showed bulbed axonal endings compared to control group (FIGS. 8A-B). Morphology of neurons, the number, length, and branching of neurites, was analyzed by Sholl analysis implemented in ImageJ [30]. Sholl analysis draws equally spaced circles from the soma and counts the number of intersections between neurites and circles to quantify neuronal morphology (FIG. 8C). Incubation with paclitaxel for one hour hampered elongation of axons (FIG. 8A, 8B, 8D, 8E). Co-administration of GSSSG restored axonal elongation (FIG. 8D, >160 μm from soma, p<0.05, paclitaxel vs paclitaxel+GSSSG), as did co-administration of PTNSSS (FIG. 8E); paclitaxel tended to shorten axonal length while PTNSSS prevented the axonal shortening induced by paclitaxel. Incubation with paclitaxel also decreased the ratio of the total mitochondria length to axonal length and the number of mitochondria in axons, which is a sign of increased mitochondrial fragmentation (FIG. 9A). GSSSG prevented paclitaxel-induced decrease of the ratio of the total mitochondria length to axonal length (FIG. 9B, p<0.0001) and the number of mitochondria (FIG. 9C, p=0.0017). These results suggest that the beneficial effects of GSSSG against PIPN is mediated by prevention of axonal degeneration via preservation of mitochondrial integrity in peripheral neural axons.

### Example 7. GSSSG Attenuated Paclitaxel-Induced Increase of Superoxide Levels in Primary Cortical Neurons

We examined the change of intracellular ROS in primary cortical neurons because another conceivable effect of GSSSG is antioxidant effect [14]. After incubating primary cortical neurons with 100 nM of paclitaxel for one hour, intracellular levels of superoxide was measured by Dihydroethidium (DHE). Levels of superoxide were increased after incubation with paclitaxel (FIG. 10, p=0.01, paclitaxel vs control). Co-incubation with 10 μM of GSSSG prevented the paclitaxel-induced increase of intracellular superoxide (p=0.0114, paclitaxel vs paclitaxel+GSSSG). These results suggest that GSSSG attenuates ROS production induced by paclitaxel.

### Example 8. GSSSG Modestly Upregulated Antioxidant Signaling in DRG

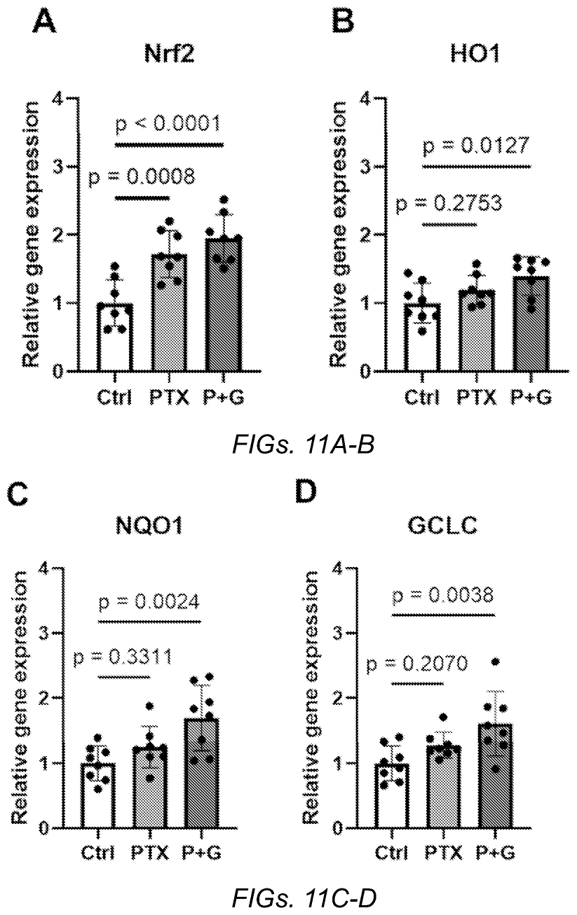

To further characterize the beneficial effects of GSSSG on PIPN, we measured mRNA levels of antioxidant proteins in lumber DRG by real-time qPCR at 2 hours after a single paclitaxel injection (16 mg/kg) with or without 50 mg/kg GSSSG administration. While both paclitaxel and paclitaxel with GSSSG treatment increased Nrf2 mRNA levels, levels of NAD (P) H Quinone Dehydrogenase 1 (NQO1) were modestly increased only in mice that received paclitaxel and GSSSG treatment (FIG. 11, p=0.0472). These results suggest that GSSSG modestly enhances Nrf-2-dependent antioxidant signaling in DRG after paclitaxel treatment.

### Example 9. GSSSG Did not Affect Anti-Tumor Effects of Paclitaxel in a Human Breast Cancer Cell Line

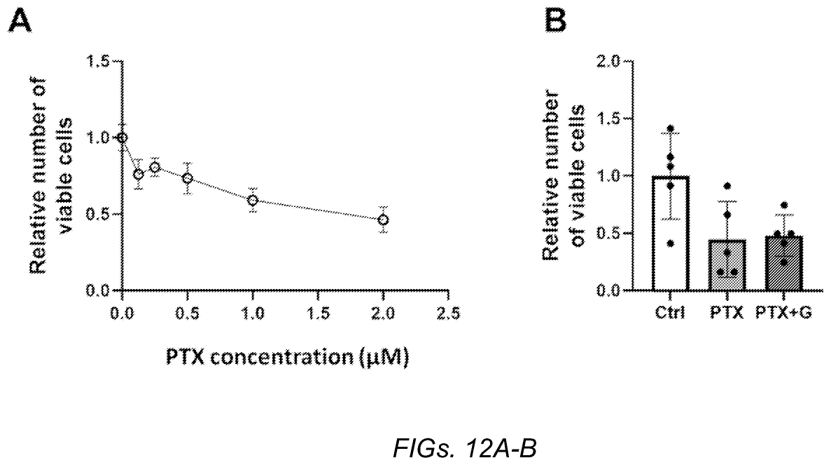

Since several studies show that enhanced antioxidant effect contributes to the resistance of cancer cells to chemotherapy [31, 32], we assessed whether GSSSG affects anti-tumor effect of paclitaxel. We examined anti-tumor effect of paclitaxel using MDA-MB-231 human breast cancer cells. After incubation with paclitaxel for 24 hours, viability of MDA-MB-231 cells was analyzed by LDH Cytotoxicity Detection assay (FIG. 12A). We found that IC50 of paclitaxel for MDA-MB-231 cells was 1.66 μM. Based on these results, we applied 2 μM of paclitaxel and 10 μM of GSSSG to MDA-MB-231 for 24 hours to examine whether co-administration of GSSSG alters cytotoxic effects of paclitaxel against MDA-MB-231 cells. After 24 hours of incubation, the number of alive MDA-MB-231 cells decreased by approximately half with or without GSSSG (PTX vs PTX+GSSSG: 44.9% vs 48.3% of control cells) (FIG. 12B). The statistical test did not show a difference more than defined 10% margin (mean difference: 0.034, 90% confidence interval: −0.280 to 0.357). These results indicate that GSSSG does not affect anti-tumor effect of paclitaxel.

### REFERENCES

1. Wilson, B. E., et al., Estimates of global chemotherapy demands and corresponding physician workforce requirements for 2018 and 2040: a population-based study. Lancet Oncol, 2019. 20(6): p. 769-780.
2. Seretny, M., et al., Incidence, prevalence, and predictors of chemotherapy-induced peripheral neuropathy: A systematic review and meta-analysis. Pain, 2014. 155(12): p. 2461-2470.
3 Dougherty, P. M., et al., Taxol-induced sensory disturbance is characterized by preferential impairment of myelinated fiber function in cancer patients. Pain, 2004. 109(1-2): p. 132-42.
4 Colvin, L. A., Chemotherapy-induced peripheral neuropathy: where are we now? Pain, 2019. 160 Suppl 1: p. S1-S10.
5 Yang, I. H., et al., Compartmentalized microfluidic culture platform to study mechanism of paclitaxel-induced axonal degeneration. Exp Neurol, 2009. 218(1): p. 124-8.
6. Pease-Raissi, S. E., et al., Paclitaxel Reduces Axonal Bclw to Initiate IP3R1-Dependent Axon Degeneration. Neuron, 2017. 96(2): p. 373-386 e6.
7 Chine, V. B., et al., Targeting Axon Integrity to Prevent Chemotherapy-Induced Peripheral Neuropathy. Mol Neurobiol, 2019. 56(5): p. 3244-3259.
8 Koltzenburg, M., Neural mechanisms of cutaneous nociceptive pain. Clin J Pain, 2000. 16(3 Suppl): p. S131-8.
9 Flatters, S. J. L. and G. J. Bennett, Studies of peripheral sensory nerves in paclitaxel-induced painful peripheral neuropathy: evidence for mitochondrial dysfunction. Pain, 2006. 122(3): p. 245-257.
10. Xiao, W. H., et al., Mitochondrial abnormality in sensory, but not motor, axons in paclitaxel-evoked painful peripheral neuropathy in the rat. Neuroscience, 2011. 199: p. 461-9.
11. McCormick, B., et al., Mito VitE, a mitochondria-targeted antioxidant, limits paclitaxel-induced oxidative stress and mitochondrial damage in vitro, and paclitaxel-induced mechanical hypersensitivity in a rat pain model. Br J Anaesth, 2016. 117(5): p. 659-666.
12. Duggett, N. A., et al., Oxidative stress in the development, maintenance and resolution of paclitaxel-induced painful neuropathy. Neuroscience, 2016. 333: p. 13-26.
13. Zheng, H., W. H. Xiao, and G. J. Bennett, Functional deficits in peripheral nerve mitochondria in rats with paclitaxel- and oxaliplatin-evoked painful peripheral neuropathy. Exp Neurol, 2011. 232(2): p. 154-61.
14. Ida, T., et al., Reactive cysteine persulfides and S-polythiolation regulate oxidative stress and redox signaling. Proc Natl Acad Sci USA, 2014. 111(21): p. 7606-11.
15. Benchoam, D., et al., Acidity and nucleophilic reactivity of glutathione persulfide. J Biol Chem, 2020. 295(46): p. 15466-15481.
16. Wall, P. D., Vigilance in defense of animal welfare. International Association for the Study of Pain. Pain, 1993. 54(3): p. 239.
17. Chen, L. H., et al., Targeting interleukin-20 alleviates paclitaxel-induced peripheral neuropathy. Pain, 2020. 161(6): p. 1237-1254.
18. Gonzalez-Cano, R., et al., Up-Down Reader: An Open Source Program for Efficiently Processing 50% von Frey Thresholds. Front Pharmacol, 2018. 9: p. 433.
19. Chaplan, S. R., et al., Quantitative assessment of tactile allodynia in the rat paw. J Neurosci Methods, 1994. 53(1): p. 55-63.
20. Bannon, A. W. and A. B. Malmberg, Models of nociception: hot-plate, tail-flick, and formalin tests in rodents. Curr Protoc Neurosci, 2007. Chapter 8: p. Unit 8 9.
21. Lauria, G., et al., European Federation of Neurological Societies/Peripheral Nerve Society Guideline on the use of skin biopsy in the diagnosis of small fiber neuropathy. Report of a joint task force of the European Federation of Neurological Societies and the Peripheral Nerve Society. Eur J Neurol, 2010. 17(7): p. 903-12, e44-9.
22. Ghnenis, A. B., et al., Toluidine Blue Staining of Resin-Embedded Sections for Evaluation of Peripheral Nerve Morphology. J Vis Exp, 2018(137).
23. Sleigh, J. N., S. J. West, and G. Schiavo, A video protocol for rapid dissection of mouse dorsal root ganglia from defined spinal levels. BMC Res Notes, 2020. 13(1): p. 302.
24. Hamid, H. A., et al., Polysulfide stabilization by tyrosine and hydroxyphenyl-containing derivatives that is important for a reactive sulfur metabolomics analysis. Redox Biol, 2019. 21: p. 101096.
25. Zhang, T., et al., Enhanced Cellular Polysulfides Negatively Regulate TLR4 Signaling and Mitigate Lethal Endotoxin Shock. Cell Chem Biol, 2019. 26(5): p. 686-698 e4.
26. Takata, T., et al., Methods in sulfide and persulfide research. Nitric Oxide, 2021. 116: p. 47-64.
27. Perner, C. and C. L. Sokol, Protocol for dissection and culture of murine dorsal root ganglia neurons to study neuropeptide release. STAR Protoc, 2021. 2(1): p. 100333.
28. Marutani, E., et al., A novel hydrogen sulfide-releasing N-methyl-D-aspartate receptor antagonist prevents ischemic neuronal death. J Biol Chem, 2012. 287(38): p. 32124-35.
29. Gadgil, S., et al., A systematic summary and comparison of animal models for chemotherapy induced (peripheral) neuropathy (CIPN). PLOS One, 2019. 14(8): p. e0221787.
30. Ferreira, T. A., et al., Neuronal morphometry directly from bitmap images. Nat Methods, 2014. 11(10): p. 982-4.
31. Homma, S., et al., Nrf2 enhances cell proliferation and resistance to anticancer drugs in human lung cancer. Clin Cancer Res, 2009. 15(10): p. 3423-32.
32. Cho, J. M., et al., Role of the Nrf2-antioxidant system in cytotoxicity mediated by anticancer cisplatin: implication to cancer cell resistance. Cancer Lett, 2008. 260(1-2): p. 96-108.
33. Szabo, C., Hydrogen sulphide and its therapeutic potential. Nat Rev Drug Discov, 2007. 6(11): p. 917-35.
34. Trecarichi, A. and S. J. L. Flatters, Mitochondrial dysfunction in the pathogenesis of chemotherapy-induced peripheral neuropathy. Int Rev Neurobiol, 2019. 145: p. 83-126.
35. Cirrincione, A. M., et al., Paclitaxel-induced peripheral neuropathy is caused by epidermal ROS and mitochondrial damage through conserved MMP-13 activation. Sci Rep, 2020. 10(1): p. 3970.
36. Akaike, T., et al., Cysteinyl-tRNA synthetase governs cysteine polysulfidation and mitochondrial bioenergetics. Nat Commun, 2017. 8(1): p. 1177.
37. de Beus, M. D., J. Chung, and W. Colon, Modification of cysteine 111 in Cu/Zn superoxide dismutase results in altered spectroscopic and biophysical properties. Protein Sci, 2004. 13(5): p. 1347-55.
38. Shinkai, Y., et al., Reactive Sulfur Species-Mediated Activation of the Keap1-Nrf2 Pathway by 1,2-Naphthoquinone through Sulfenic Acids Formation under Oxidative Stress. Chem Res Toxicol, 2015. 28(5): p. 838-47.
39. Smith, G. A., et al., Glutathione S-Transferase Regulates Mitochondrial Populations in Axons through Increased Glutathione Oxidation. Neuron, 2019. 103(1): p. 52-65 e6.
40. Ding, C., et al., Activation of the CaMKII-Sarm1-ASK1-p38 MAP kinase pathway protects against axon degeneration caused by loss of mitochondria. Elife, 2022. 11.
41. Baloh, R. H., Mitochondrial dynamics and peripheral neuropathy. Neuroscientist, 2008. 14(1): p. 12-8.
42. Cuadrado, A., et al., Transcription Factor NRF2 as a Therapeutic Target for Chronic Diseases: A Systems Medicine Approach. Pharmacol Rev, 2018. 70(2): p. 348-383.
43. Fidanboylu, M., L. A. Griffiths, and S. J. Flatters, Global inhibition of reactive oxygen species (ROS) inhibits paclitaxel-induced painful peripheral neuropathy. PLOS One, 2011. 6(9): p. e25212.
44. Bessaguet, F., et al., Neuroprotective effect of angiotensin II type 2 receptor stimulation in vincristine-induced mechanical allodynia. Pain, 2018. 159(12): p. 2538-2546.
45. Mo, M., et al., Prevention of paclitaxel-induced peripheral neuropathy by lithium pretreatment. FASEB J, 2012. 26(11): p. 4696-709.
46. Todd, A. J., Neuronal circuitry for pain processing in the dorsal horn. Nat Rev Neurosci, 2010. 11(12): p. 823-36.
47. Hwang, B. Y., et al., Gender differences in paclitaxel-induced neuropathic pain behavior and analgesic response in rats. Korean J Anesthesiol, 2012. 62(1): p. 66-72.
48. Scripture, C. D., W. D. Figg, and A. Sparreboom, Peripheral neuropathy induced by paclitaxel: recent insights and future perspectives. Curr Neuropharmacol, 2006. 4(2): p. 165-72.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for the treatment, or reduction of risk, of a disorder associated with peripheral neuropathy in a subject, the method comprising administering a therapeutically or prophylactically effective amount of a composition comprising glutathione trisulfide (GSSSG), pantethine trisulfide (PTN-SSS), or lipoic acid trisulfide (LA-SSS) to a subject in need thereof, optionally comprising administering the GSSSG orally or nasally.

2. The method of claim 1, further wherein the composition comprising GSSSG is prepared by dissolving a crystalline form of GSSSG in buffered saline at pH 3-6.

3. The method of claim 1, wherein the disorder is chemotherapy induced peripheral neuropathy (CIPN).

4. The method of claim 3, wherein the CIPN is paclitaxel-induced peripheral neuropathy (PIPN).

5. The method of claim 3, comprising administering an effective amount of a composition comprising GSSSG, PTN-SSS, or LA-SSS within a few minutes to hours before or after administration of a dose of chemotherapy.

6. The method of claim 3, comprising administering an effective amount of a composition comprising GSSSG, PTN-SSS, or LA-SSS every day for the first one or two weeks of a chemotherapy regimen.

7. The method of claim 1, wherein the disorder is diabetic neuropathy, Guillain-Barré syndrome neuropathy, chronic inflammatory demyelinating polyneuropathy, post herpetic neuralgia, or peripheral neuropathy caused by-hepatitis B, hepatitis C, myeloma, lymphoma, or kidney disease.

8.-14. (canceled)


SEQUENCE LISTING (10 sequences)

SEQ ID NO: 1 - DNA, 21 bp, synthetic construct
  source 1..21: mol_type = other DNA
tcctcagcagaacaggaacag

SEQ ID NO: 2 - DNA, 21 bp, synthetic construct
  source 1..21: mol_type = other DNA
cctccaaaggatgtcaatcaa

SEQ ID NO: 3 - DNA, 20 bp, synthetic construct
  source 1..20: mol_type = other DNA
aagccgagaatgctgagttc

SEQ ID NO: 4 - DNA, 23 bp, synthetic construct
  source 1..23: mol_type = other DNA
gccgtgtagatatggtacaagga

SEQ ID NO: 5 - DNA, 21 bp, synthetic construct
  source 1..21: mol_type = other DNA
aggatgggaggtactcgaatc

SEQ ID NO: 6 - DNA, 22 bp, synthetic construct
  source 1..22: mol_type = other DNA
aggcgtccttccttatatgcta

SEQ ID NO: 7 - DNA, 19 bp, synthetic construct
  source 1..19: mol_type = other DNA
ggacaaaccccaaccatcc

SEQ ID NO: 8 - DNA, 23 bp, synthetic construct
  source 1..23: mol_type = other DNA
gttgaactcagacatcgttcctc

SEQ ID NO: 9 - DNA, 20 bp, synthetic construct
  source 1..20: mol_type = other DNA
cggctaccacatccaaggaa

SEQ ID NO: 10 - DNA, 18 bp, synthetic construct
  source 1..18: mol_type = other DNA
gctggaattaccgcggct